(19) United States
(12) Patent Application Publication
(10) Pub. No.: US 2011/0180065 A1
(43) Pub. Date: Jul. 28, 2011

(54) ENDOTRACHEAL TUBE PROTECTOR

(75) Inventors: Julius Hajgato, Shanty Bay (CA); Kenneth Noseworthy, Barrie (CA)

(73) Assignee: SOUTHMEDIC INCORPORATED, Barrie (CA)

(21) Appl. No.: 13/081,923

(22) Filed: Apr. 7, 2011

Related U.S. Application Data
(63) Continuation-in-part of application No. 11/865,411, filed on Oct. 1, 2007.

(51) Int. Cl. A61M 16/04

(52) U.S. Cl. 128/200.26

(57) ABSTRACT

An endotracheal tube protector for use with an endotracheal tube comprises an elongated tubular member having a first end configured to extend out of a patient's mouth when in use and a second end configured to enter the throat of the patient. The tubular member has openings in the first and second ends and defines a substantially cylindrical cavity to hold an endotracheal tube. The tubular member has a longitudinal slit to allow the walls to separate for radial expansion of the cavity. The wall thickness is non-constant, with a thin-walled region which flexes to permit the walls to separate, and a thick-walled region which resists crushing forces acting on the protector. A pair of opposed bosses may extend longitudinally adjacent to the slit, to transmit a clamping force onto the endotracheal tube when contacted by the patient's jaw.

### CROSS-REFERENCE TO RELATED APPLICATION(S)

The present application is a Continuation-in-part of application Ser. No. 11/865,411, filed on Oct. 1, 2007, the contents of which are incorporated herein by reference, in their entirety.

### FIELD OF THE INVENTION

The present invention relates to medical equipment, specifically to a protector for an endotracheal tube or similar device which enters a patient's mouth. More particularly, the invention relates to a bite block configured to secure endotracheal tubes of different diameters while protecting the tube from pinching or damage from the clamp forces applied by the jaw of a patient.

### BACKGROUND OF THE INVENTION

Endotracheal tubes are used to provide a clear passage for air to the lungs in cases of injury or airway blockage or to provide a passage through which medical probes or instruments can be inserted such that damage to the tissues of the patient is minimized. Such endotracheal tubes often are used in conjunction with a secondary air tube that leads to an inflatable bulb which functions to seal the space between the tracheal wall and the endotracheal tube so that saliva and/or other bodily fluids are prevented from entering the lungs.

One type of endotracheal tube protector is referred to as a “bite block”, and is used to secure the tube to a holder that can be retained in the patient's mouth. A bite block normally protects the tube from damage or pinching resulting from when the patient inadvertently bites on the protector with too much force. In some cases, the patient can apply a sufficiently powerful bite that can damage or pinch the tube if the bite block is insufficiently rigid and robust. A bite block also protects the delicate tissues of the patient from abrasions caused by entry of the endotracheal tube into the oral cavity. As well, when a supplemental air tube is fed into the patient's mouth, it is desirable to protect this tube from the patient's bite.

Prior art devices typically comprise a hollow cylinder having an elongate slot extending the length thereof, for example as described by Briggs, III in U.S. Pat. No. 5,649,534. The cylinder wall of this structure is described as being essentially rigid to prevent crushing of the tube in the event of a powerful bite from the patient, but can be slightly resilient to grip the tube. Another example is described in U.S. Pat. No. 5,829,430 to Islavia, which relates to a bite block having a generally rectangular external cross-sectional configuration, with a central slot and inwardly-projecting flanges which contact the tube.

There is a need for a bite block that accommodates a range of endotracheal tube sizes, while at the same time firmly gripping and protecting the tube from damage, and which is easily and disengaged engaged with the tube.

### SUMMARY OF THE INVENTION

It is an object of the invention to provide a bite block that can be used as an endotracheal tube protector capable of accommodating endotracheal tubes of different sizes, while lessening the discomfort experienced by the intubated patient.

In one aspect, the invention relates to an endotracheal tube protector comprising a tubular member having a first end for extending out of a patient's mouth and an opposed second end which is placed in the mouth of the patient. The tubular member has openings at both ends and defines a substantially cylindrical cavity with an interior wall configured to retain an endotracheal tube. The tubular member consists of a pair of arcuate walls that converge at their distal edges to define a longitudinal slit, which permits the walls to separate to provide radial expansion of the cavity to accommodate a range of tube sizes. The tubular member is formed from a flexible resilient material which permits the walls to separate for radial expansion, with the resiliency of the walls serving to urge them inwardly when radially expanded so as to apply a compressive force to grip the endotrachial tube within the cavity.

In one aspect, the tubular member further includes a pair of opposed axially-extending bosses adjacent to the longitudinal slit. The opposed bosses project into the cavity such that they contact and transmit a clamping force to the endotracheal tube when the endotracheal tube protector is contacted by the upper teeth of the patient given that the endotracheal tube protector is appropriately oriented such that the slit can face toward the roof of the patient's mouth, or rotated 90° from that position. The bosses also serve to space the endotracheal tube from the cavity wall to provide a gap between the endotracheal tube and the interior wall to accommodate a secondary air tube for inflation of a bulb. Protection of the secondary air tube prevents it from being bitten or otherwise crushed in the oral cavity. The bulb protects the distal end of the endotracheal tube from blockage by bodily fluids and to prevent bodily fluids from collecting in the lungs of the patient.

The endotracheal tube protector may include a third axially extending boss which projects into the cavity at a position opposing the longitudinal slit.

The endotracheal tube protector may also include a base along the length of the tubular member opposite the longitudinal slit, having a flat external surface for contacting the patient's lower teeth and for resting on the patient's tongue.

To lessen patient discomfort, the tubular member may also be tapered towards the second end such that the radius of the tubular member is progressively reduced to provide less contact and pressure against the throat and the roof of the patient's mouth. The endotracheal tube protector may also have an inwardly cut-away portion positioned and configured for allowing the endotracheal tube to bend outwardly at an angle from the endotracheal tube protector, thus providing more flexibility for the endotracheal tube to be positioned optimally in context of the endotracheal intubation system.

The invention also relates to a method for positioning an endotracheal tube into the trachea of a patient. This method is performed by providing the endotracheal tube protector as described above, positioning the endotracheal tube into the cavity of the endotracheal tube protector, and inserting the back end of the endotracheal tube protector into the oral cavity of the patient such that the longitudinal slit is oriented towards the roof of the patient's oral cavity and orienting the endotracheal tube protector such that the patient's upper and lower teeth provide clamping force onto the endotracheal tube.

The invention also relates to an endotracheal intubation system which can provide either gas delivery or which can introduce a probe or medical instrument into the trachea of a patient. The system includes the endotracheal tube protector as described above, an endotracheal tube, and one or more of a system for gas delivery, a probe or a medical instrument. The probe or medical instrument are configured for slidable entry through the endotracheal tube. The system may also include an air tube for inflation of a bulb which is designed to protect the distal end of the endotracheal tube from blockage by bodily fluids and to prevent bodily fluids from collecting in the lungs of the patient.

According to another aspect, the invention relates to a bite block for an endotracheal tube, in which the bite block has an essentially cylindrical tubular configuration comprising an elongate base, a pair of opposing resilient arcuate sidewalls projecting from said base along the proximal edge of each wall and an internal cavity to receive an endotracheal tube. For directional reference, central longitudinal axis is defined between opposing ends of the protector, with vertical and horizontal bisecting planes which meet along said axis. The sidewalls converge at their distal edges (opposed to the base) on opposing sides of the vertical plane to define an openable slot therebetween. The sidewalls comprise arcuate inner surfaces defining an essentially cylindrical cavity to confine an endotracheal tube therein, and outer surfaces. The inner and outer surfaces define a non-constant wall thickness. Each wall comprises a relatively thin-walled region adjacent to the base to permit the walls to flex outwardly away from each other for receiving and gripping said tube, and a relatively thick-walled region spaced from the base to resist crushing forces. The expression “relatively” herein refers to the aspect wherein the thin-walled regions are thinner between the inner and outer surfaces than the thick-walled regions.

In another aspect, the thick-walled regions on opposing ones of said walls are located directly opposed to each other and define opposing shoulders, which may be located either midway between the base and slit on the horizontal plane, or above the horizontal plane. The walls may taper in thickness between said shoulder and said base, wherein the wall thicknesses of the thin and thick-walled regions are selected whereby substantially all of the flexure of said wall occurs within said thin-walled region when said walls are separated. The thin-walled regions on opposing ones of the sidewalls are likewise directly opposed to each other.

According to another aspect the bite block further comprises at least one secondary bore within the base extending parallel to the longitudinal axis for retaining a secondary tube therein parallel to the endotracheal tube. The secondary bore may comprise a tubular structure projecting from said base, on an opposed side thereof from the cavity.

The present invention will now be further described by a non-limiting detailed description of embodiments thereof. In the present specification, directional references are presented purely for ease of description and do not limit the scope of the invention. As described herein, the tube holder is positioned in a generally horizontal position, such as would be the case when gripped within a patient's mouth his head in an upright position. It will be seen that the device may be rotated into any orientation. As well, unless otherwise expressed, any dimensions herein are provided merely by way of examples of the invention.

The terms “endotracheal tube protector” and “bite block” are used in a generally interchangeable fashion herein, unless specifically stated otherwise or the context otherwise suggests. Although the present invention is described for use as a protector for an endotrachial tube, it will be seen that the invention may be adapted for similar medical uses.

### BRIEF DESCRIPTION OF THE DRAWINGS

FIG. 1 is a cross sectional view of the endotracheal tube protector in use.

FIG. 2 is a side view of the endotracheal tube protector.

FIG. 3 is a top view of the endotracheal tube protector.

FIG. 4 is a cross sectional view of the endotracheal tube protector along line 3-3 of FIG. 6.

FIG. 5 is a further cross sectional view of the endotracheal tube protector along line 3-3 of FIG. 6, including cross sections of the air line and the endotracheal tube.

FIG. 6 is a perspective view of the endotracheal tube protector.

FIG. 7 is a perspective view of the endotracheal tube protector.

FIG. 8 is a perspective view of a bite block according to a second embodiment of the invention.

FIG. 9 is a plan view, from above, of the second embodiment.

FIG. 10 is a front elevational view of the second embodiment.

FIG. 11 is a side elevational view of the second embodiment.

FIG. 12 is a front elevation view of the second embodiment, showing the wide walls in an open, spread-apart configuration.

FIG. 13 is a perspective view of the second embodiment, showing two bite blocks engaged to an endotracheal tube and an air tube, respectively.

FIG. 14 depicts the second embodiment in use with a patient.

### DETAILED DESCRIPTION

The endotracheal tube protector 10, also referred to as bite block 10, is configured to retain an endotracheal tube and to provide a robust but comfortable holder for the patient. Bite block 10 is configured to protect an endotracheal tube from inadvertent biting by a patient.

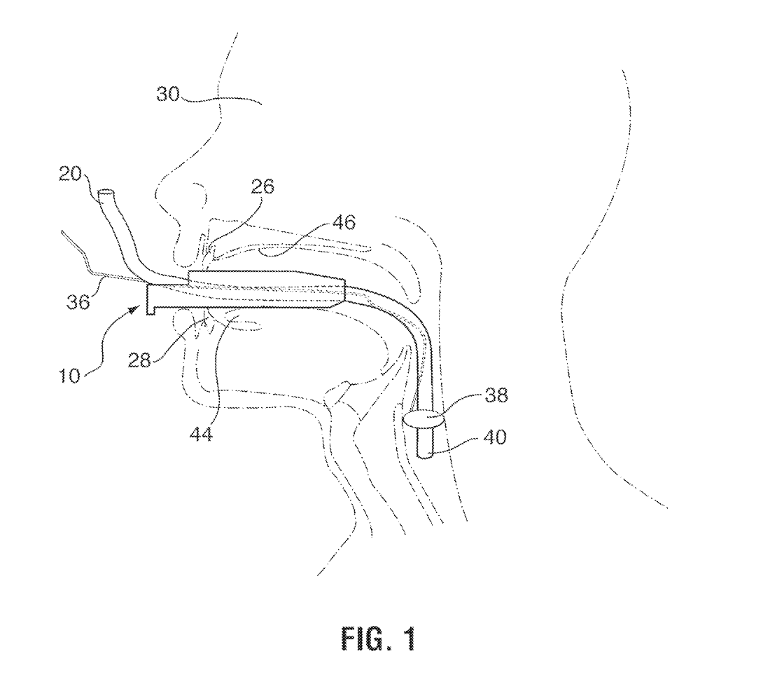

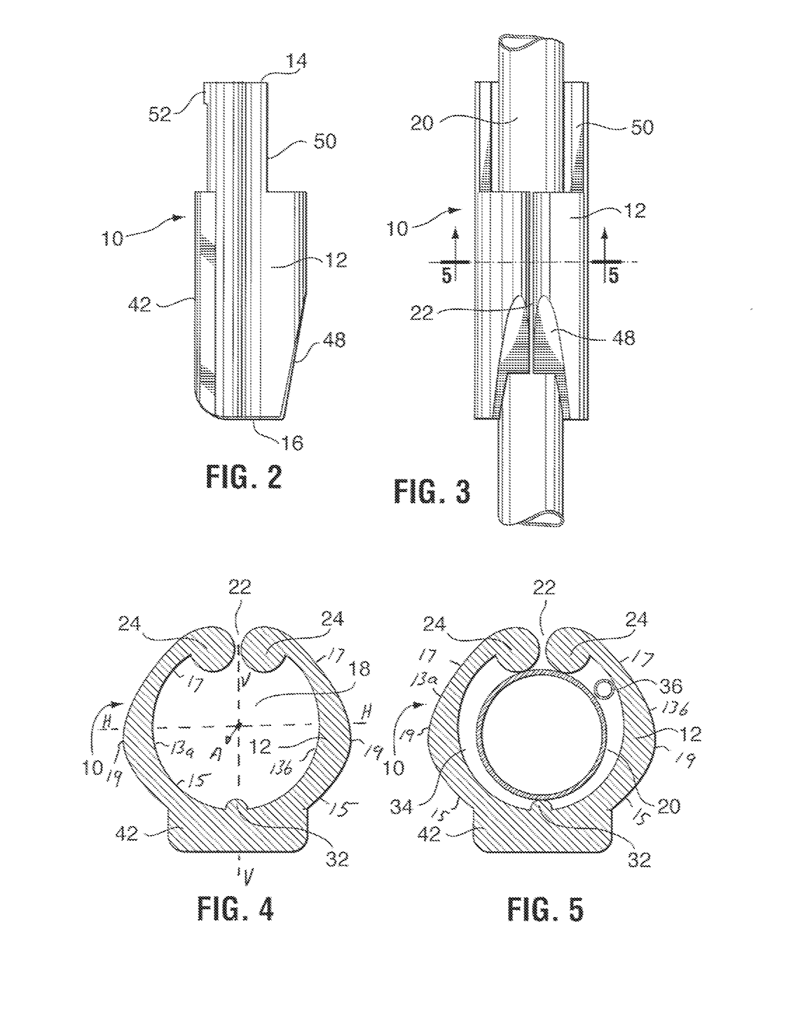

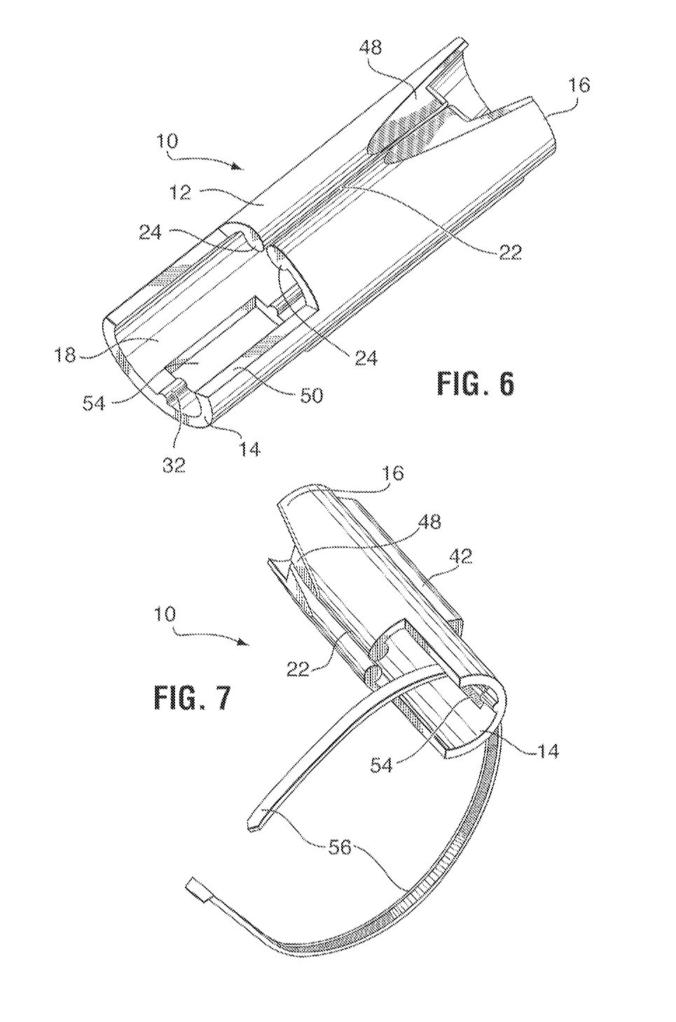

As seen in FIGS. 1-7, a first embodiment of protector 10 comprises an elongated tubular member 12 formed of resilient material and consisting of an integral, monolithic member. Tubular member 12 has a first end 14 and a second opposed end 16 with an elongate central axis A extending therebetween. The first end 14 extends outwardly from the mouth of a patient when in use, while the second end 16 is located inside the patient's mouth. Tubular member 12 defines a substantially cylindrical cavity 18, extending lengthwise within the device. A conventional endotracheal tube 20 can be retained within cavity 18 with sufficient gripping force to effectively prevent slippage of the tube 20 during normal use. Tubular member 12 has a longitudinal slit 22 to permit radial expansion of the cavity 18 and which divides member 12 into two opposing arcuate side walls 13a and 13b. Slit 22 parallels axis A. The resiliency of tubular member 12 provides the function whereby walls 13a and b can be separated to radially expand cavity 18 to accommodate a range of sizes of tube 20. When urged apart, walls 13 apply a resulting compressive force against a tube retained therein.

For purposes of directional reference, vertical and horizontal planes V and H bisect member 12 and intersect along axis A, as seen in FIG. 4. Walls 13a and b are symmetrically disposed on either side of the vertical plane V.

The exposed uppermost edges of walls 13a and 13b form a pair of opposed bosses 24 extending axially on either side of vertical plane V. Slit 22 extends between bosses 24. Bosses 24 project into cavity 18 and contact tube 20 so as to transmit a clamping force onto tube 20, in particular when the patient bites downwardly onto member 12.

Bite block 10 also includes a third boss 32 projecting into the cavity 18 at a position directly opposing longitudinal slit 22, for contacting tube 20. Boss 32 is located on vertical place V. Third boss 32 projects upwardly into cavity 18 and extends longitudinally the length thereof parallel to the central axis.

Opposed bosses 24 and the third boss 32 are configured to provide, when tube 20 is installed, a gap 34 between tube 20 and the interior surface of the cavity 18. The gap is of sufficient size to accommodate and protect an air tube 36 for inflation of a bulb 38 (see FIG. 1). Bulb 38 protects the distal end 40 of endotracheal tube 20 from blockage by bodily fluids and to prevent bodily fluids from collecting in the lungs of the patient.

Tubular member 12 includes a flat base 42 extending the full length of tubular member 12 at a position directly opposed to slit 22. Base 42 protrudes downwardly has a substantially flat lower surface for contacting the patient's lower teeth and/or tongue 44. Base 42 has arcuate upper surface facing the interior of cavity 18 that is continuous with the inside surface of walls 13a and b. Cavity 18 thus forms a cylindrical surface to engage a similarly shaped tube 20 having a circular sectional configuration.

Bosses 24 are in contact or near contact with each other when the walls are in their relaxed, unflexed position. Thus, slit 22 is narrow or even non-existent until the walls are flexed. In the normal operation of the device, the walls are not highly flexed, such that tube 20 is almost entirely encircled by the interior surface of cavity 18 to provide a grip on tube 20.

Seen in cross-section, each sidewall 13 has a tapered, non-constant wall thickness. In the first embodiment, and as seen particularly in FIGS. 4 and 5, walls 13a and b each comprise a relatively thin-walled lower region 15 located adjacent to base 42, at the proximal edge of the wall, and a second thin-walled upper region 17 at the distal region of the wall adjacent to boss 24. A relatively thick-walled intermediate region 19 is located between regions 15 and 17. The respective regions taper continuously into each other and thus there is no sharp division between the various regions. As a result of the differential wall thicknesses, a majority of the flexure of walls 13 occurs within the respective regions 15 and 17 when walls 13a and b are separated. Thick-walled region 19 is relatively inflexible, which tends to prevent deformation of member 12 when a vertical crushing force is applied by the patient's jaws against member 12. When walls 13a and b are separated, the countervailing biasing force urging them together is thus largely applied by the thin, more flexible regions 15 and 17.

Tubular member 12 has a taper 48 in a direction extending toward the second end such that the cross section of the tubular member 12 is progressively reduced to provide less contact and pressure, when in use, against the roof 46 of the patient's mouth, thereby improving the comfort of the patient.

First end 14 of the tubular member 12 is provided with an inwardly cut-away portion 50 extending towards the second end 16. The cut-away portion is positioned and configured to allow the endotracheal tube 20, when in position, to extend outwardly from the tubular member 12 at an angle thereto.

The first end 14 of the endotracheal tube protector 10 has a slot 54 which is configured to receive a means for securing the endotracheal tube to said endotracheal tube protector. The means for securing can be a cable tie 56, elastic band, or other such device. The cable tie 56 is configured to encircle and secure the endotracheal tube 20 to the endotracheal tube protector 10 to prevent the endotracheal tube 20 from moving and dislodging from the endotracheal tube protector 10 or sliding further down the patient's trachea during use.

Endotracheal tube protector 10 further includes a handle 52 extending from the first end 14 in a substantially perpendicular direction with respect to a plane defined by the length of the tubular member 12. Handle 52 is configured for grasping and facilitating removal of the endotracheal tube protector 10 from the mouth of the patient.

Endotracheal tube protector 10 is moulded in a single integral and monolithic structure from resilient flexible plastic material. The resiliency and flexibility of walls 13a and b allows them to be spread apart to fit, in one version, endotracheal tubes of diameters ranging from about 6 millimeters to about 9.5 millimeters within cavity 18. In another version, bite block 10 is configured to fit pediatric tubes ranging from about 4 to 6.5 millimeters. The material from which the endotracheal tube protector is moulded has a Shore A durometer scale hardness from about 60 to about 90. This hardness of the material is selected to provide sufficient friction against the patient's mouth and throat that a means for securing the endotracheal tube protector from sliding down into the patient's throat is not required. Protector 10 can be fabricated from a material that can be heated for sterilizing and which is relatively inexpensive for disposable use.

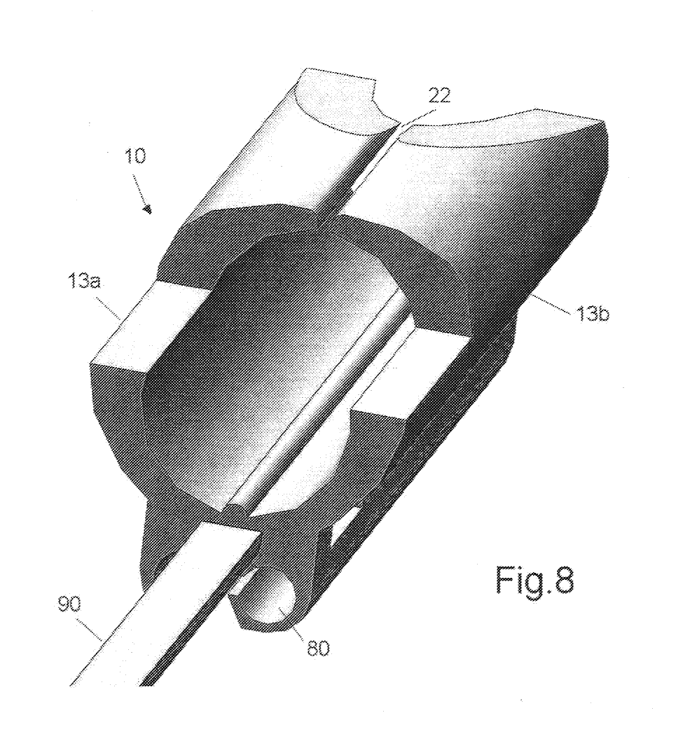

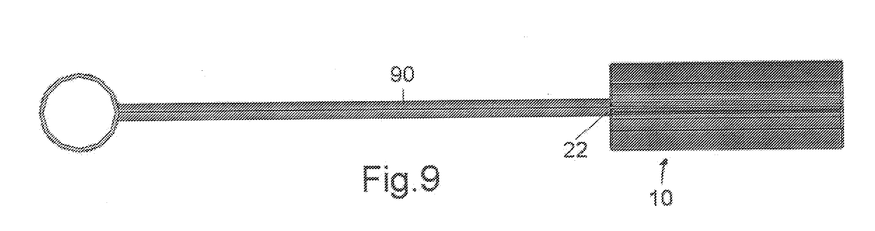

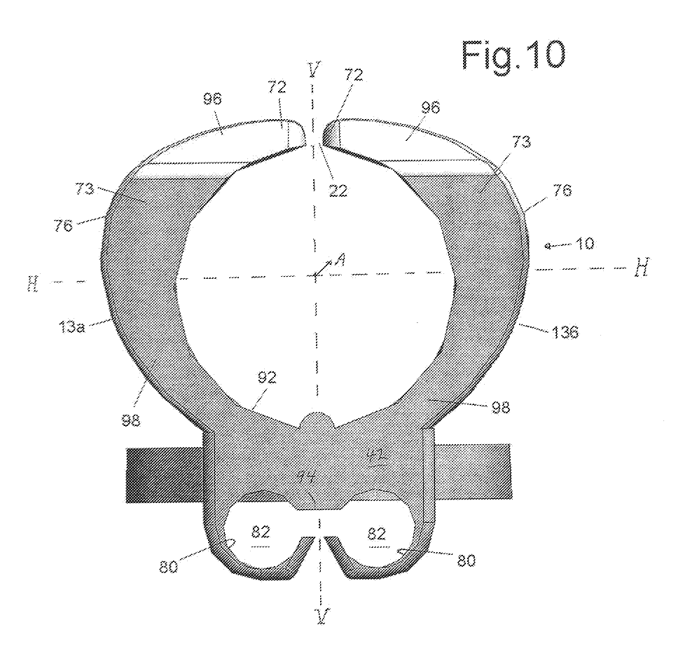

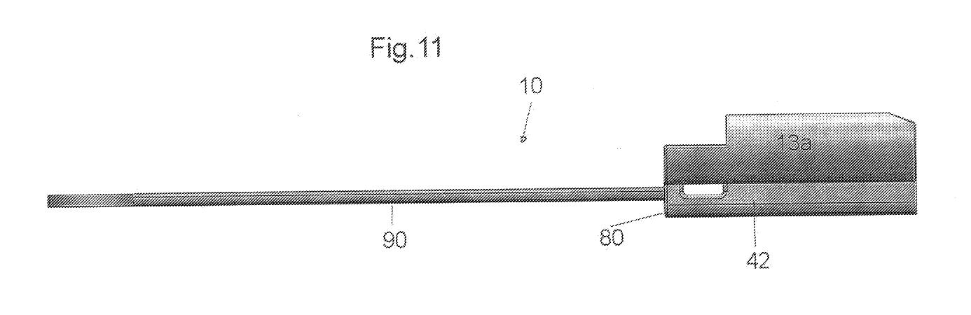

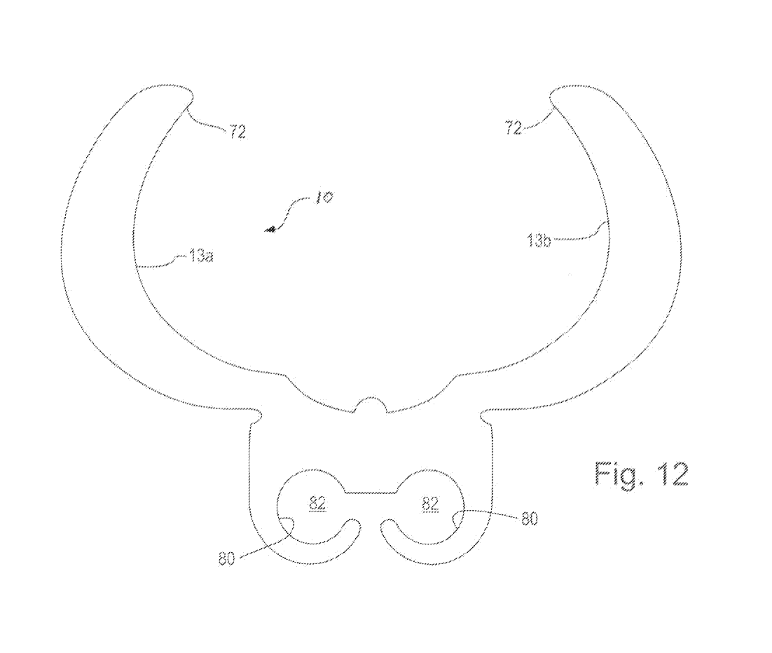

A further embodiment of bite block 10 is illustrated in FIGS. 8-14. Walls 13a and b in the second embodiment do not include a boss along their distal edges but instead each taper towards a relatively thin lip 72 (see FIG. 10). The space between respective lips 72 defines slit 22. When walls 13a and b are unflexed, slit 22 can be either relatively narrow or closed. Walls 13a and b are symmetric on either side of vertical plane V. Walls 13 have a non-constant thickness, in a similar fashion to the previous embodiment, with a curved inner surface which defines a cylindrical central cavity (essentially circular in cross-section) and a non-cylindrical outer surface (non-circular in cross section). Walls 13a and b each comprise a thick-walled middle region 73 that is located relatively close to lip 72, i.e. on the same side of bisecting plane H as lips 72. Thick-walled regions 73 define a pair of protruding shoulders 76 on the outer surfaces of walls 13a and b. Walls 13a and b become progressively thinner in either direction away from shoulders 76 towards thin-walled regions 96 and 98 adjacent to lips 72 and base 42 respectively. As with the first embodiment, the thickness of the walls has a constant taper between the thickest portion thereof at shoulders 76 and the respective thin-walled regions 96 and 98.

Base 42 comprises an arcuate inside surface 92 which is continuous with the cylindrical surface of walls 13, and a projecting external portion with a flat outer surface 94. Base 42 thus provides a substantially rigid, inflexible region which resists deformation. Thick-walled shoulder regions 73 likewise resist deformation.

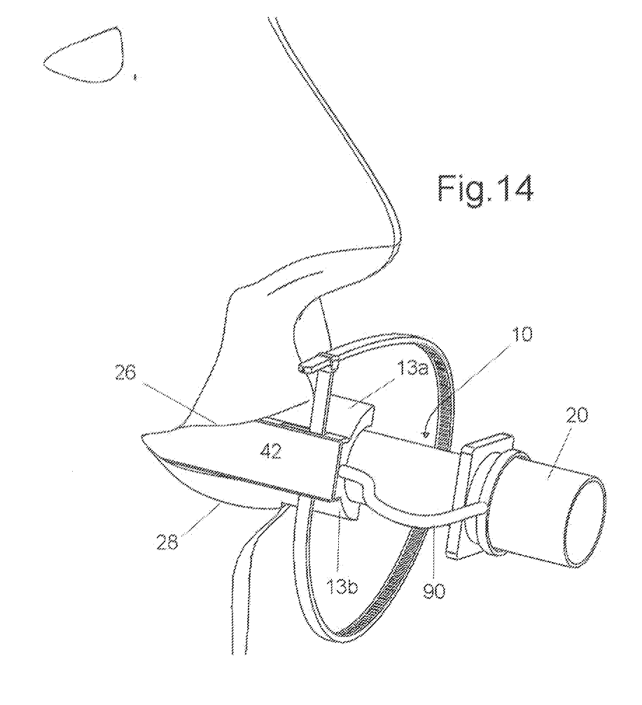

In use, the device can be rotated 90° into a sideways position, as seen in FIG. 14, wherein slit 22 is in a horizontal bisecting plane and shoulders 76 form the upper and lower contact surfaces for the patient's teeth.

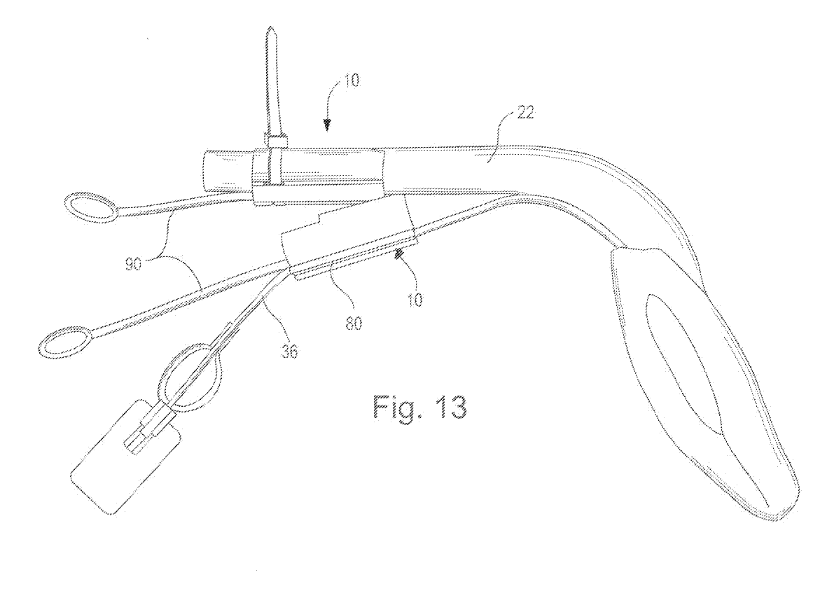

When walls 13a and b are separated to expand cavity 18 in order to receive and engage tube 20 (see FIG. 13), the majority of flexure occurs within the relatively thin-walled and flexible region 98, which can flex sufficiently to accommodate a relatively wide range of tube sizes within the expandable cavity 18. The device is then placed in the mouth of the patient and the patient's teeth can contact shoulders 76. When shoulders 76 are gripped by the patient's jaws, walls 13a and b are urged together so as to firmly grip tube 20. Depending on the tube diameter, walls 13a and b can overlap at slit 22 when urged together so as to reduce the inside diameter of cavity 18 for gripping tube 20.

Even when gripped tightly between the patient's jaws, thick-walled regions 73 resist deformation to prevent pinching or damage to tube 20. In order to serve this function, bite block 10 is fabricated from a material that is sufficiently unyielding, and with suitable wall thicknesses, to minimize the flexure of walls 13a and b while still permitting sufficient resilience to allow a range of endotrachial tubes 20 to be inserted with relative ease by separation of walls 13a and b. It is within the skill of persons skilled in the relevant art to select such a material and dimensions. By way of one non-limiting example, bite block 10 has an internal diameter of the cavity of 4 or 6 mm., and a wall thickness ranging between about 1.2 mm at thin-walled regions 15 and 17, and about 3.4 mm at shoulders 76.

The embodiment of protector 10 shown in FIGS. 8-14 further relates to an alternative means to retain the secondary air tube 36 to the protector. In this embodiment, base 42 incorporates at least one, and preferably a plurality, of secondary tube retainers formed from a pair of opposing walls 80. Walls 80 project downwardly from base 42, and are semi-circular in cross-section. Walls 80 each define a semi-enclosed bore 82, which each comprise a slot where wall 80 curves inwardly towards base 42. Walls 80, which are monolithic with bite block 10, are sufficiently thin to provide flexibility and resiliency to permit a tube 36 to be inserted into bore 82 through the slot, or alternatively by threading tube 36 lengthwise into bore 82. The resiliency of walls 80 provides a gripping force on tube 36. Tube 36 is thus generally protected from damage from the patient's teeth or pinching due to pressure from endotrachial tube 20 where tubes 36 pass through device 10. The tube retainers are located side by side and parallel to each other. Walls 80 effectively provide base 42 with a hollow construction that is rigid and crush resistant, while remaining lightweight.

Walls 80 are integral with base 42 and may project outwardly from the lower face of base 42, as illustrated. Alternatively, bores 82 may be wholly incorporated within the interior of base 42 (not shown) whereby base 42 has an essentially flat lower surface opposed to slot 22.

Bite block 10 may be used to retain at the same time an endotrachial tube 20 and one or more air tubes 36. Alternatively, two bite blocks 10 may be provided, one of which holds tube 20 and the other of which holds tube(s) 36, as seen in FIG. 13. This arrangement is possible since tube 36 is retained independently of tube 20, such that the presence of tube 20 is not required for tube 36 to be retained in the bite block. In this arrangement, the patient can be sequentially given the respective bite blocks 10 depending on which tube is in active use in the patient, such that only one tube at a time need be delivered to the patient.

In the embodiment of FIGS. 8-14, there is also shown a retainer 90 to provide a supplemental means for retaining the endotracheal tube 20 to the bite block 10. Retainer 90 is described in more detail in the present inventors' co-pending application Ser. No. 12/241,506, the contents of which are incorporated herein by reference.

Endotracheal tube protector 10 is used for intubating a patient. Such methods may be performed by positioning an endotracheal tube 20 into the central cavity of the endotracheal tube protector 10 and inserting the second end 16 of the endotracheal tube protector into the oral cavity of the patient. In one procedure, the longitudinal slit 22 is oriented towards the roof 46 of the patient's mouth. The endotracheal tube protector is then oriented such that the patient's upper 26 and lower 28 jaws can contact the upper and lower surfaces of protector 10.

As seen in FIG. 14, bite block 10 can be rotated 90° when positioned in the patient's mouth, whereby slit 22 is disposed laterally on the horizontal plane of the device.

Endotracheal tube protector 10 can be employed in combination with other components in endotracheal intubation systems. Such systems can include, for example, a gas delivery means such as an oxygen or air delivery system, or a probe or medical instrument configured for slidable entry into the endotracheal tube. The system may also include an air tube 36 for inflation of a bulb which is configured to protect the distal end of the endotracheal tube from blockage by bodily fluids and to prevent the bodily fluids from collecting in the lungs of the patient.

It will be seen that the present invention has been described by way of preferred embodiments of various aspects of the invention. However, it will be understood that one skilled in the art may readily depart from the embodiments described in detail herein, while still remaining within the scope of the invention as defined in this patent specification including the claims thereto.

1. A bite block comprising a tubular member having opposed ends and a longitudinal slot extending the full length of said wall to permit radial expansion of said member, said member comprising a resilient wall having an inner surface which defines an essentially cylindrical cavity, and an outer surface, wherein the inner and outer wall surfaces define a wall thickness which is non-constant and comprises at least one relatively thin-walled flexible region and a relatively thick-walled inflexible region to resist crushing forces applied against said bite block.

2. The bite block of claim 1 comprising two of said walls opposed to each other, wherein the thin walled regions in respective ones of said walls are directly opposed to each other and the thick-walled regions in respective ones of said walls are directly opposed to each other.

3. The bite block of claim 2 wherein said thin-walled regions in respective ones of said walls are remote from said slot and said thick-walled regions are intermediate between said thin-walled regions and said slot.

4. The bite block of claim 3 wherein said thick-walled regions in respective ones of said walls form opposing shoulders projecting from said tubular member.

5. The bite block of claim 3 wherein the thickness of said wall has a generally constant taper between said thick and thin-walled regions.

6. The bite block of claim 1 further comprising an elongate resilient rib within said cavity extending parallel to said longitudinal axis, for contacting said tube.

7. The bite block of claim 2 wherein said walls converge towards a slot directly opposed to said base, said walls being symmetric about said slot.

8. The bite block of claim 7 wherein each of said walls further comprises an elongate boss along an edge thereof facing said slot, wherein said bosses comprise the opposing sides of said slot, said bosses projecting inwardly into said cavity.

9. A bite block comprising opposing ends with a longitudinal axis extending between said ends, a base and a pair of opposing resilient arcuate walls extending from said base, said walls comprising proximal edges at said base and opposed distal edges, wherein said walls converge along their distal edges and together comprise inner surfaces defining an essentially cylindrical cavity configured to confine an endotracheal tube therein and outer surfaces, wherein the inner and outer surfaces on each said walls define a non-constant wall thickness comprising a relatively thin-walled flexible proximal region to permit the walls to flex outwardly away from each other to expand the radius of the cavity, and a relatively thick-walled inflexible region intermediate between the distal edge and the thin-walled region to resist crushing forces applied against said bite block.

10. The bite block of claim 9 wherein each of said walls comprises a second thin-walled region adjacent to said distal edge.

11. The bite block of claim 9 wherein said base comprises a flat outer surface and a curved, cradle-shaped inner surface forming part of the cylindrical cavity.

12. The bite block of claim 9 wherein the thick-walled regions on opposing ones of said walls are directly opposed to each other and define opposing shoulders.

13. The bite block of claim 12 wherein said walls have a constant taper between said shoulder and said base.

14. The bite block of claim 13 wherein the thicknesses of the thin and thick-walled regions are selected whereby substantially all of the flexure of said wall occurs within said thin-walled region when said walls are separated.

15. The bite block of claim 9 further comprising at least one secondary bore within the base extending parallel to the longitudinal axis for retaining a secondary tube therein parallel to the endotracheal tube.

16. The bite block of claim 15 comprising two of said secondary bores in side by side relationship.

17. The bite block of claim 15 wherein said secondary bore comprises a semi-circular wall projecting from said base.

18. The bite block of claim 9 wherein the distal edges of said walls are in contact or near contact when the walls are unflexed.

19. The bite block of claim 9 further comprising at least one endotracheal tube.

20. The bite block of claim 16 further comprising at least one endotracheal tube and at least one secondary air tube configured to fit within said secondary bore.
